(19) United States
(12) Patent Application Publication
(10) Pub. No.: US 2010/0227907 A1
(43) Pub. Date: Sep. 9, 2010

(54) Method of Treating Pain by Using Opioids and CAMKIV Inhibitors

(76) Inventor: Min Zhuo, Toronto (CA)

Correspondence Address: VALENTINE A COTTRILL;SUSAN TANDAN, 50 QUEEN STREET NORTH, STE. 1020, P.O. BOX 2248, KITCHENER, ON, N2H6M2, CA

(21) Appl. No.: 12/086,338

(22) PCT Filed: Dec. 14, 2006
(86) PCT No.: PCT/CA2006/002034

§ 371 Date: Jun. 11, 2008

Related U.S. Application Data
(60) Provisional application No. 60/751,251, filed on Dec. 16, 2005.

(51) Int. Cl. A61K 31/713; A61P 25/04

(52) U.S. Cl. 514/44 A

(57) ABSTRACT

A method of treating chronic pain in a mammal is provided comprising the steps of inhibiting CaMKIV in the mammal and administering to the mammal a therapeutically effective amount of an opioid.

### FIELD OF THE INVENTION

The present invention relates to methods of treating pain. In particular, the present invention relates to methods of treating pain by regulating CaMKIV levels.

### BACKGROUND OF THE INVENTION

Calcium-calmodulin dependent protein kinase IV (CaMKIV) activates the cAMP response-element binding protein (CREB) by phosphorylating it at Ser 133 (Deisseroth et al., 1996; Deisseroth et al., 1998). Phosphorylated CREB recruits the transcriptional co-activator CBP (CREB binding protein), which leads to the activation of CRE (cAMP response element) containing promoters and ultimately to gene expression (Ginty, 1997; Matthews et al., 1994; Soderling, 1999; Wei et al., 2002). CaMKIV is a calcium-dependent protein kinase that is detected in both the nuclei and cytoplasm of neurons, and is the only protein kinase to phosphorylate CREB that is detected predominately in the nuclei of neurons (Jensen et al., 1991; Kang et al., 2001; Nakamura et al., 1995). A recent study reported that CaMKIV KO mice show a defect in fear memory, while responses to acute noxious stimuli were normal compared to wild-type mice (Wei et al., 2002). Learning, memory, and drug addiction have certain intracellular signaling cascades in common and depend on the transcription factor CREB (Kandel, 2001; Nestler, 2001b; Nestler, 2002).

Numerous studies implicate CREB in drug addiction (Blendy and Maldonado, 1998; Guitart et al., 1992; Hyman, 1996; Maldonado et al., 1996; Walters and Blendy, 2001). Western blot analysis showed an increase in CREB or phosphorylated CREB (pCREB) expression in morphine-tolerant animals (Gao et al., 2004; Li and Clark, 1999) and CREB mutant mice displayed less severe withdrawal symptoms following cessation of chronic morphine treatment (Maldonado et al., 1996). CREB has also been implicated in the positive and negative reinforcing properties of drugs of abuse (Barrot et al., 2002; Carlezon et al., 1998; Walters and Blendy, 2001). Additionally, the Mu opioid receptor (MOR), a G-protein-coupled receptor that primarily mediates the physiological actions of morphine, contains a CRE element and was shown to be activated through CREB-mediated pathways (Lee and Lee, 2003; Nestler, 1997). A dominant negative form of CREB was able to decrease MOR expression in culture (Lee and Lee, 2003). One prevalent theory suggests that tolerance develops from the decreased coupling of the MOR to an inhibitory G-protein (Christie et al., 1987; Nestler, 2001a; Sim et al., 1996). Additionally, the MOR can be desensitized through phosphorylation by several protein kinases (PKA, PKC, CaMKII and G protein coupled receptor kinases), which leads to the development of tolerance and dependence (Liu and Anand, 2001; Mestek et al., 1995).

A number of studies have reported a role for protein kinases in opioid tolerance (Eitan et al., 2003; Terman et al., 2004; Ueda et al., 2001; Zeitz et al., 2001). Inhibition of protein kinase C (PKC) blocks the development of acute opioid tolerance (Narita et al., 1995) and PKCγ mutant mice showed reduced tolerance to the analgesic effects of morphine (Zeitz et al., 2001). Another study showed that calcium-calmodulin kinase II (CaMKII) activity was increased in the spinal cord of tolerant rats and a CaMKII inhibitor was able to reverse the already established analgesic tolerance (Wang et al., 2003).

Another emerging theory on the development of opioid tolerance centers on the role of the delta opioid receptors (DORs). The development of opioid tolerance can be attenuated using DOR antagonists (Abdelhamid et al., 1991; Fundytus et al., 1995) and is abolished in DOR knockout mice (Zhu et al., 1999). Previous studies showed that prolonged treatment with morphine increased the potency of DOR agonists and promoted targeting of DORs to the cell surface of neurons in the dorsal horn in a process that involves the activation of the MOR (Cahill et al., 2001; Morinville et al., 2003). Future work is needed to determine the molecular mechanisms responsible for the morphine-induced targeting of DOR to the neuronal membrane.

Several bodies of evidence suggest that CaMKIV may play an important role in the behavioral and molecular responses to morphine. CaMKIV is unique in its ability to phosphorylate CREB in the nuclei of neurons and may play a role in the transcriptional modifications following morphine exposure. It would be desirable, thus, to determine the role of CaMKIV as it relates to opioid use and pain management in order that more efficacious methods of pain control may be developed.

### SUMMARY OF THE INVENTION

It has now been found that, in the absence of the protein kinase, CaMKIV, there is reduced analgesic tolerance to chronic opioid treatment. Accordingly, pain resulting from, for example, chronic conditions, can be treated using typically administered opioid dosages in the presence of CaMKIV inhibition. This obviates the need to use undesirable increased opioid dosages as a result of opioid tolerance that has developed due to recurring or continual opioid use, as well as the need to look to alternative pain treatments.

Thus, in one aspect of the present invention, a method of treating pain in a mammal is provided comprising the steps of inhibiting CaMKIV in the mammal and administering to the mammal a therapeutically effective amount of an opioid.

In another aspect of the invention, a method of treating pain is provided comprising administration of an opioid in combination with an inhibitor of CaMKIV.

In another aspect of the present invention, a method of preventing, or at least reducing, the development or occurrence of opioid analgesic tolerance in a mammal during opioid treatment is provided comprising the step of inhibiting CaMKIV.

In a further aspect of the present invention, a pharmaceutical composition is provided comprising a therapeutically effective amount of an opioid in combination with a CaMKIV inhibitor.

In yet a further aspect of the present invention, an article of manufacture is provided comprising packaging material and a pharmaceutical composition, wherein the composition comprises a therapeutically effective amount of an opioid in combination with a CaMKIV inhibitor, and wherein the packaging material is labelled to indicate that the composition is useful to treat chronic pain.

These and other aspects of the invention will become evident in view of the description and drawings in which:

### BRIEF DESCRIPTION OF THE DRAWINGS

FIG. 1 graphically illustrates that response latencies using a hotplate test are similar between morphine-treated wild-type and CaMKIV KO mice (A), while CaMKIV KO mice exhibit less analgesic tolerance following chronic morphine treatment than wild-type counterparts in both hot plate (B) and tail flick (C) tests;

FIG. 2 (A/B) graphically illustrates that locomotor activity is similar between CaMKIV KO and wild-type mice following acute (A) and chronic (B) morphine treatment;

FIG. 2C illustrates that CaMKIV KO mice show a reduced preference for the morphine paired side in the conditioned place preference test;

FIG. 2(D-H) graphically illustrates that there is no difference in withdrawal behaviors between CaMKIV KO and wild-type mice;

FIG. 3 shows that chronic morphine-induced increase in pCREB expression is absent in CaMKIV KO mice (A/B) while levels of MOR(C) and phosphorylated (p)MOR (D) in CaMKIV KO mice are comparable to that in wild-type mice;

FIG. 4 (A-C) illustrates the results of an immunohistochemical analysis of pCREB and MOR expression in the lumbar enlargement of the spinal cord in saline and morphine treated CaMKIV KO and wild-type mice;

FIG. 5 graphically illustrates that Mu opioid receptor uncoupling is significantly decreased in wild-type mice compared to CaMKIV KO mice after chronic morphine;

FIG. 6 illustrates representative examples of sIPSCs in SG neurons in dorsal horn from control and morphine-treated wild-type (A) and CaMKIV KO (B) mice, and graphically illustrates sIPSC frequency (C-left) and average inhibition rates (C-right) after acute morphine treatment as well as sIPSC frequency (D-left) and average inhibition rates (D-left) after chronic morphine;

FIG. 7 illustrates the subcellular distribution of immunoreactive DOR in wild-type and CaMKIV KO mice both treated and untreated with morphine;

FIG. 8A illustrates effect of ionomycin on CREB activity in HEK 293 cells transiently expressing CaMKIV;

FIG. 8B illustrates the dose-dependent inhibitory effect of KN62 and KN93 on CaMKIV transiently expressed in HEK293 cells;

FIG. 9A is a western blot analysis of CaMKIV expression in control and cultured neurons treated with siRNA #1 and #2; and

FIG. 9B graphically illustrates CaMKIV expression in cultured neurons treated siRNA #1 and #2.

### DETAILED DESCRIPTION OF THE INVENTION

A novel method of treating pain in a mammal is provided. The method includes the steps of inhibiting CaMKIV in the mammal in combination with administration of a therapeutically effective amount of an opioid. The method is effective to reduce the occurrence of opioid analgesic tolerance and, thus, is particularly useful for the treatment of chronic pain in which ongoing analgesia is required.

The term “CaMKIV” refers to calcium-calmodulin dependent protein kinase IV. CaMKIV activates the cAMP response-element binding protein (CREB) by phosphorylation at the serine positioned at 133 (Ser¹³³). For the purposes of the present invention, CaMKIV is not restricted with respect to a particular amino acid sequence, and encompasses any mammalian CaMKIV kinase that functions to phosphorylate CREB. It will be appreciated by those of skill in the art that functionally equivalent variants of mammalian CaMKIV may exist which retain CREB phosphorylating activity, but which vary in amino acid sequence. For example, CaMKIV may be defined by amino acid sequences set out in NCBI accessions #NM-009793 and #NM-00744, the contents of each of which are incorporated herein by reference.

In accordance with the present method, inhibition of CaMKIV prevents, or at least reduces, the occurrence of analgesic tolerance in prolonged opioid treatment. Inhibition of CaMKIV may occur at the nucleic acid level, for example using anti-sense, snp or siRNA technologies. Alternatively, CaMKIV may be inhibited at the protein level. Inhibitors of CaMKIV can be identified using assays, for example, in which CREB expression is measured.

CaMKIV-encoding nucleic acid molecules, such as that described in Nature 420 (6915), 520-562 (2002) (Accession # NC 000084) may be used to prepare antisense oligonucleotides against CaMKIV which may be therapeutically useful to inhibit expression of the CaMKIV gene. Accordingly, antisense oligonucleotides that are complementary to a nucleic acid sequence encoding CaMKIV according to the invention are also provided. The term “antisense oligonucleotide” as used herein means a nucleotide sequence that is complementary to a target CaMKIV nucleic acid sequence.

The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halouracil, 5-halocytosine, 6-azathymine, pseudo-uracil, 4-thiouracil, 8-haloadenine, 8-aminoadenine, 8-thioladenine, 8-thiolalkyl adenines, 8-hydroxyl adenine and other 8-substituted adenines, 8-halo guanines, 8-amino guanine, 8-thiol guanine, 8-thiolalkyl guanines, 8-hydrodyl guanine and other 8-substituted guanines, other aza and de-aza uracils, thymidines, cytosines, adenines, or guanines, 5-tri-fluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. For example, phosphorothioate bonds may link only the four to six 3′-terminal bases, may link all the nucleotides or may link only 1 pair of bases.

The antisense oligonucleotides of the invention may also comprise nucleotide analogs that may be better suited as therapeutic or experimental reagents. An example of an oligonucleotide analogue is a peptide nucleic acid (PNA) in which the deoxribose (or ribose) phosphate backbone in the DNA (or RNA), is replaced with a polymide backbone which is similar to that found in peptides (P. E. Nielson, et al Science 1991, 254, 1497). PNA analogues have been shown to be resistant to degradation by enzymes and to have extended lives in vivo and in vitro. PNAs also form stronger bonds with a complementary DNA sequence due to the lack of charge repulsion between the PNA strand and the DNA strand. Other oligonucleotide analogues may contain nucleotides containing polymer backbones, cyclic backbones, or acyclic backbones. For example, the nucleotides may have morpholino backbone structures (as described in U.S. Pat. No. 5,034,506). Oligonucleotide analogues may also contain groups such as reporter groups, a group for improving the pharmacokinetic properties of an oligonucleotide, or a group for improving the pharmacodynamic properties of an antisense oligonucleotide. Antisense oligonucleotides may also incorporate sugar mimetics as will be appreciated by one of skill in the art.

Antisense nucleic acid molecules may be constructed using chemical synthesis and enzymatic ligation reactions using procedures known in the art based on the CaMKIV nucleic acid and/or amino acid sequence information such as that provided. The antisense nucleic acid molecules of the invention, or fragments thereof, may be chemically synthesized using naturally occurring nucleotides or variously modified nucleotides designed to increase the biological stability of the molecules or to increase the physical stability of the duplex formed with mRNA or the native gene, e.g. phosphorothioate derivatives and acridine substituted nucleotides.

The antisense sequences may also be produced biologically using technologies including recombinant technology. Thus, the antisense oligonucleotides may be introduced into tissues or cells via a vector (such as a recombinant plasmid, phagemid, retroviral vectors, adenoviral vectors and DNA virus vectors) which produces the antisense sequences under the control of a high efficiency regulatory region, the activity of which may be determined by the cell type into which the vector is introduced.

Anti-sense oligonucleotides may be administered to a mammal using physical techniques such as microinjection. The antisense oligonucleotides may be directly administered in vivo using microinjection, for example, or may be administered using transfected cells in vitro which are then administered in vivo.

In another embodiment, siRNA technology can be applied to prevent expression of CAMKIV. Application of nucleic acid fragments such as siRNA fragments that correspond with regions in CaMKIV and which selectively target the CaMKIV gene may be used to block CaMKIV expression resulting in reduced analgesic tolerance. Such blocking occurs when the siRNA fragments bind to the CaMKIV gene thereby preventing translation of the gene to yield functional CaMKIV.

SiRNA, small interfering RNA molecules, corresponding to the CaMKIV gene are made using well-established methods of nucleic acid syntheses including automated systems. Since the structure of the CaMKIV gene is known, fragments of RNA that correspond therewith can readily be made. The effectiveness of selected siRNA to block CaMKIV activity can be confirmed using a CaMKIV-expressing cell line. Briefly, selected siRNA is incubated with a CaMKIV-expressing cell line under appropriate growth conditions. Following a sufficient reaction time, i.e. for the siRNA to bind with CaMKIV DNA to result in decreased expression of the CaMKIV DNA, the reaction mixture is tested to determine if such decreased expression has occurred. Suitable siRNA will prevent processing of the CaMKIV gene to yield functional CaMKIV. This can be detected by assaying for CaMKIV function in the reaction mixture, for example, CREB activity.

Regions from the CaMKIV gene from which selective siRNA can be derived include, for example, the central region of the gene. Examples of siRNA fragments determined to have use in the present method include, but are not limited to, the sense strand 5′-GGAUGAGUCCUCCAUGUUCtt-3′ and antisense strand 5′-GAACAUGGAGGACUCAUCCtt-3′. It will be appreciated by one of skill in the art that selected siRNA fragments, such as those identified, may be modified to yield functionally equivalent siRNA fragments that retain the ability to inhibit expression of CaMKIV. Suitable modifications that may be made to a selected siRNA fragment to yield a functionally equivalent fragment include, for example, addition, deletion or substitution of one or more of the nucleotide bases therein, provided that the modified siRNA retains it ability to bind to the targeted CaMKIV gene. Selected siRNA fragments may additionally be modified in order to yield fragments that are more desirable for use. For example, siRNA fragments may be modified to attain increased stability thereof.

The present method of treating pain includes, in addition to inhibiting CaMKIV, administration of an opioid. Use of opioids to treat pain is well-known in the art. An opioid is any compound, peptide or otherwise, which possesses some affinity for at least one of the opioid receptor subtypes, such as the mu opioid receptor. Opioids are classified as full agonist, partial agonist and mixed agonist/antagonist based on their characteristics. Full agonist opioids do not have a ceiling to their analgesic efficacy and will not reverse or antagonize the effects of other full agonist opioids when administered simultaneously. Full agonist opioids include morphine, codeine, oxycodone, hydrocodone, methadone, levorphanol, and fentanyl. Partial agonist opioids exhibit relatively low intrinsic efficacy at the opioid receptor in comparison to full opioid agonists and display a ceiling effect to analgesia. An example of a partial agonist is buprenorphine. Mixed agonist-antagonists, in contrast to full agonists, have an analgesic ceiling and block opioid analgesia at one type of opioid receptor (mu) or are neutral at this receptor while simultaneously activating a different opioid receptor (kappa). Examples of mixed agonist-antagonists in clinical use include pentazocine, butorphanol tartrate, dezocine, and nalbuphine hydrochloride. As would be appreciated by one of skill in the art, patients receiving full opioid agonists should not be given a mixed agonist-antagonist as it may precipitate withdrawal syndrome and increase pain. Full agonist opioids, partial agonist opioids and mixed agonist-antagonists are suitable for use in a method of the present invention, particularly those targeting the mu opioid receptor.

Applicable dosages of opioids to treat pain are well-established and, in accordance with the present invention, will generally be in the range of 1 mg-1500 mg adult dose per day, administered orally or parenterally. As will be appreciated, dosages and dosage forms will vary with the individual and condition being treated.

Thus, in the present method of treating pain in a mammal, CaMKIV is inhibited by administration to the mammal of an appropriate antisense oligonucleotide(s), siRNA fragment(s) or by administration of a chemical CaMKIV inhibitor, followed by, or in conjunction with, administration of a selected opioid as described above. I

In another aspect of the present invention, a method of reducing the occurrence or development of opioid analgesic tolerance during opioid treatment is provided. As set out above, this method involves inhibiting CaMKIV to an extent appropriate to reduce analgesic tolerance to the opioid.

In another aspect of the present invention, an article of manufacture is provided. The article comprises packaging material and a pharmaceutical composition. The composition comprises a therapeutically effective amount of an opioid in combination with a CaMKIV inhibitor and the packaging material is labeled to indicate that the composition is useful to treat chronic pain.

The packaging material may be any suitable material generally used to package pharmaceutical agents including, for example, glass, plastic, foil and cardboard.

Embodiments of the invention are described by reference to the following specific examples which are not to be construed as limiting.

### EXAMPLES

### Experiment #1

### CaMKIV Knock-Out Effect on Opioid Tolerance

### Methods

### Animals

CaMKIV KO mice were derived as described (Wei et al., 2002) and bred for several generations (F8-F12) on C57B1/6 background. Control wild-type mice were adult male (8-12 weeks old) C57B1/6 mice from Charles River. At the conclusion of experiments, animals were humanely killed by an overdose of inhaled anesthetic (halothane). The animals were housed on a 12 h:12 h light:dark cycle with food and water available ad libitum. All mouse protocols are in accordance with NIH guidelines and were approved by the Animal Care and Use Committee at the University of Toronto. No visual difference between wild-type and CaMKIV KO mice is noticeable, and experiments were performed blind.

### Open Field Activity Monitor

To record horizontal locomotor activity, the Activity Monitor system from Med Associates (43.2×43.2×30.5 cm; MED-associates, St. Albans, Vt.) was used. Briefly, this system uses paired sets of photo beams to detect movement in the open field and movement is recorded as beam breaks. The open field is placed inside an isolation chamber with dim illumination and a fan. Each subject was placed in the center of the open field and activity was measured for 60 minutes.

### Conditioned Place Preference

A chamber with two distinct contextual environments (different walls, floor and smell) was used (MED-associates, St. Albans, Vt.). On the first day of testing, animals were allowed to freely explore both sides of the chamber for 30 min and data were used to separate animals into groups of approximately equal bias. For the next eight days, each animal was given either 10 mg/kg morphine or an equivalent volume of saline on alternating days in distinct sides of the chamber. The animals were confined to the specific side of the chamber for 30 min. After conditioning, all animals were injected with saline and allowed to freely explore both sides of the chamber for 30 min. Place preference was defined as an increase in the time spent in the morphine-paired side after conditioning as compared to before.

### Hot Plate and Tail Flick Tests

The hot plate consists of a thermally-controlled metal plate (55° C.), surrounded by four Plexiglass walls (Columbia Instruments; Columbus, Ohio). The time between placement of the animal on the plate and the licking or lifting of a hindpaw is measured with a digital timer. Mice were removed from the hot plate immediately after the first response and a cut-off time of 30 seconds was imposed to prevent tissue damage. The spinal tail flick reflex (Columbia Instruments; Columbus, Ohio) was evoked by focused, radiant heat applied to the underside of the tail and a cut-off time of 10 seconds was imposed to prevent tissue damage. Response latencies are reported as a percentage of maximal possible effect (MPE) [(response latency-baseline response latency)/(cut off latency-baseline response latency)*100].

### Acute Tolerance

Acute analgesic tolerance to morphine was induced as described (Bohn et al., 2000). On the first day, animals received either 100 mg/kg morphine (s.c.) (Sigma, St. Louis, Mo.) or an equivalent volume of saline. Twenty-four hours after the initial dose, all animals received 10 mg/kg of morphine (s.c) and hot plate and tail flick responses were recorded after 60 minutes.

### Chronic Tolerance

To study the development of analgesic tolerance generated by continued morphine administration, mice were injected once a day for seven days with 10 mg/kg morphine (s.c.). Open field locomotor activity was recorded for 1 hr after injection followed by determination of hot plate and tail flick response latencies.

### Morphine Withdrawal

Withdrawal behaviors were scored seven days after receiving bi-daily injections of escalating doses of morphine. The dosing scheme was as follows: Day 1-dose curve (1, 5, 10, 20 and 40 mg/kg, s.c.), Day 2-20 mg/kg×2, Day 3-40 mg/kg×2, Day 4-60 mg/kg×2, Day 5-80 mg/kg×2, Day 6-100 mg/kg×2, Day 7-100 mg/kg. Naloxone (1 mg/kg, s.c.) was used to precipitate withdrawal 2 hours after the last morphine injection. For the scoring of withdrawal behaviors, mice were placed individually in plastic containers and scored for 30 minutes. Wet-dog shakes, paw tremors, jumping, mastication, diarrhea, and hot plate response latencies were scored by two researchers blind to the animal's genotype.

### Western Blot Analysis

Spinal cords were dounce-homogenized (18 strokes) in 10 volumes of 10 mM Tris-Cl (pH 7.4), 320 mM Sucrose, and 1% SDS. The tissue homogenates were centrifuged at 21,000×g at 4° C. for 5 min. Equal amounts of protein from tissue samples were separated by 4-12% gradient SDS-PAGE (Invitrogen), followed by Western blotting with anti-MOR-1 (1:1000), anti-pCREB (1:1000) (Santa Cruz Biotechnology) or anti-phosphoserine antibody (for immunoprecipitated samples, 1:1000) (Sigma) followed by revelation with ECL (Amersham). Membranes (BioTrace PVDF, Pall Co.) were routinely stripped and re-probed with a monoclonal antibody for actin (1:5000, Sigma) blotting to ensure equal protein loading.

For immunoprecipitation, synaptosomal membrane fractions (LP1) were prepared as previously described (Dunah and Standaert, 2001) and solubilized using 1% SDS in TEVP buffer: 10 mM Tris-HCl, pH 7.4, 1 mM EDTA, 1 mM EGTA, 1× protease inhibitor cocktail (Sigma), and 1× phosphatase inhibitor cocktail 1 and 2 (Sigma). The solubilized proteins were diluted 20 fold with modified RIPA buffer (50 mM Tris-HCl, pH 7.4, 1% NP-40, 0.25% Na-deoxycholate, 150 mM NaCl, 1 mM EDTA, 1 mM PMSF), and incubated with 50 μl of protein G-agarose beads precoupled with anti-MOR-1 antibody (Santa Cruz Biotechnology) for 3 h at 4° C. The reaction mixtures were then washed three times and eluted by boiling in sample loading buffer and subjected to western blot as described above. Equal amounts of synaptosomal membrane fractions were used for the western blotting.

### Immunostaining

Spinal cord slices at the lumbar region were prepared using standard histological methods. Slices were incubated with rabbit anti-pCREB IgG (1:250) (Calbiochem) or rabbit anti-MOR IgG (1:500) (ImmunoStar Incorporated) at 4° C. overnight. Followed by donkey anti-rabbit IgG conjugated with Alexa 488 (1:500) (Molecular Probes) for one hour at room temperature. Measurements were made from at least three randomly selected non-contiguous sections in each mouse (3-4 mice per treatment group), observed from coded slides and averaged so that each animal has a mean value for regional immunoreactivity. The total number and density of positive cells were measured in dorsal horn layers I-II and ventral horn layers VI-IX. All data were expressed as a ratio between cell number or density and unit area. Images were obtained with an Olympus microscope. Data were analyzed with ImagePro software (Media-Cybrenetics,).

### [³⁵S] GTPγS Binding Assay

Mouse spinal cord tissue was homogenized by a dounce homogenizer in membrane preparation buffer (50 mM Tris.Cl pH 7.4, 1 mM EGTA, 3 mM MgCl₂). The homogenate was centrifuged at 500 g for 10 min at 4° C. and the supernatant was centrifuged at 48,000 g for 20 min at 4° C. (Chen and Pan, 2003). The supernatant was discarded and the crude membrane pellet was re-suspended in assay buffer (50 mM Tris.Cl pH7.4, 0.2 mM EGTA, 3 mM MgCl₂, 100 mM NaCl, 50 μM GDP). 10 μg of membrane protein was incubated in 50 pM [³⁵S]GTPγS (1,250 Ci/mmol, NEN, Boston, Mass.) and 10-10,000 nM DAMGO at 30° C. for 1 hr (Johnson et al., 2003). Binding was terminated by rapid filtration over GF/B filter using vacuum manifold (Schleicher & Schuell). The filter was washed 3 times in 50 mM Tris.Cl pH7.4, 5 mM MgCl₂ and then the bound radioactivity was measured with a liquid scintillation counter (Pharmacia Wallac 1410).

### Whole-Cell Patch Clamp Recordings in Young Spinal Cord Slices

Postnatal mice (12-19 days old) were anesthetized with 1-2% halothane and the thoracic and lumbar spinal cord were dissected out and put into pre-equilibrated artificial cerebrospinal solution fluid (ACSF) at 1-4° C. The spinal cord was placed into the shallow groove of an agar block, and several drops of 20% gelatin were placed on the low-thoracic and high-lumbar segment to half-embed the spinal cord. The agar block, together with the half-embedded spinal cord, was glued on the stage of a vibratome with cyanoacrylate adhesive, and was immersed in the ice-cold ACSF. Then, transverse slices of the lumber spinal cord (350 μm) were prepared. Slices were transferred to a room temperature submerged recovery chamber with oxygenated (95% O₂ and 5% CO₂) solution containing (in mM): NaCl, 124; NaHCO₃, 25; KCl, 4.4; KH₂PO₄, 1; CaCl₂, 2; MgSO₄, 2; glucose, 10. After one hr recovery, slices were placed in a recording chamber on the stage of an Axioskop 2FS microscope (Zeiss) equipped with infrared DIC optics for patch clamp recordings. Substantia gelatiosa (SG) could be identified as a translucent band capping the dorsal part of the gray matter under the microscope. Spontaneous inhibitory postsynaptic currents (sIPSCs) were recorded with an Axon 200B amplifier (Axon Instruments, CA). Recording electrodes (3-5 MΩ) contained a pipette solution composed of (in mM): Cs-gluconate, 120; NaCl, 5; MgCl₂ 1; EGTA, 0.5; Mg-ATP, 2; Na₃GTP, 0.1; HEPES, 10; pH 7.2; 280-300 mOsmol. Access resistance was 15-30 MΩ and was monitored throughout the experiment. Data were discarded if access resistance changed more than 15% during an experiment. The membrane potential was held at −70 mV throughout the experiment. A holding potential of +10 mV was used to record spontaneous inhibitory postsynaptic currents (sIPSCs).

### Electron Microscopy Studies

To determine the DOR plasma membrane density following chronic morphine treatment in WT and CaMKIV KO mice, animals were pretreated with 5, 8, 10, 15 mg/kg morphine sulfate (s.c., Q 12 h) and subcellular localization of DOR was assessed 8 hours after the last morphine injection (n=3 per group). Tissues obtained from non-treated mice were examined to determine whether the sub-cellular distribution of DOR was different in WT compared to KO mice. Results were compared between 4 groups: WT untreated, WT morphine-treated, KO untreated and KO morphine-treated. Immunohistochemistry was performed as previously described (Morinville et al., 2003). Briefly, mice were anaesthetized with sodium pentobarbital and perfused through the left ventricle with 50 ml of heparin followed by 30 ml of a mixture of 3.75% acrolein and 2% paraformaldehyde (PFA) in 0.1M Phosphate Buffer (PB), pH 7.4, and then by 250 ml of 2% PFA in PB. Spinal cords were removed by laminectomy and post-fixed in 2% PFA for 30 minutes at 4° C. Transverse sections (50 μm) were cut using a vibrating microtome and collected in PB. Sections were incubated with 1% sodium borohydride in PB followed by incubation in cryoprotectant solution prior to snap-freezing with isopentane, liquid nitrogen, and thawing in PB. Sections were then incubated in 3% normal goat serum (NGS) in Tris Buffered Saline (TBS). They were then incubated for 48 hours at 4° C. with a DOR antiserum (1:5000, Chemicon, Temecula, Calif.) in TBS containing 0.5% NGS. Control sections were processed in the absence of primary antibody. Sections were then incubated with colloidal gold (1 nm)-conjugated goat anti-rabbit IgG (1:50 AuroProbe One GAR, Amersham Pharmacia Biotech, Inc., Baie D'Urfé, QC) diluted in 0.1 M phosphate buffered saline (PBS), pH 7.4, containing 2% gelatin and 8% BSA. After thorough washing, sections were fixed with 2% glutaraldehyde and immunogold deposits were enhanced by silver intensification (IntenSE M Silver Enhancement Kit, Amersham Pharmacia Biotech, Inc.). Sections were post-fixed with 2% OsO₄, dehydrated in graded alcohols, embedded in Epon, and sectioned with an ultramicrotome. Ultrathin sections (80 nm) were counter-stained with uranyl acetate and lead citrate prior to observation with a Hitachi electron microscope.

For quantification of the distribution of immunolabelled DOR, a minimum of 50 immunopositive dendrites in cross section from the dorsal horn (lamina III-V) of the spinal cord were counted for each animal. Profiles were considered to be immunolabelled if three or more gold particles were present over them. Gold particles were classified as being intracellular or plasma membrane-associated. They were considered to be membrane-associated only if in actual contact with the plasma membrane; gold particles in close proximity to, but not touching, the plasma membrane were considered to be intracellular. The percentage of membrane-associated to total gold particles for each dendrite was calculated and then percentage of all dendrites per condition was averaged for each animal. Data was analyzed using Prism 3.02 (Graph Pad Software Inc.). Statistical significance was ascertained using ANOVA followed by a Tukey's MCT.

### Data Analysis and Statistics

Statistical comparisons were made using the t-test, or one way or two way ANOVA (Student-Newmann-Keuls test was used for post hoc comparison). All data is represented by the mean+/−S.E.M. In all cases, p<0.05 is considered statistically significant.

### Results

### Morphine Induced Analgesia in CaMKIV KO Mice

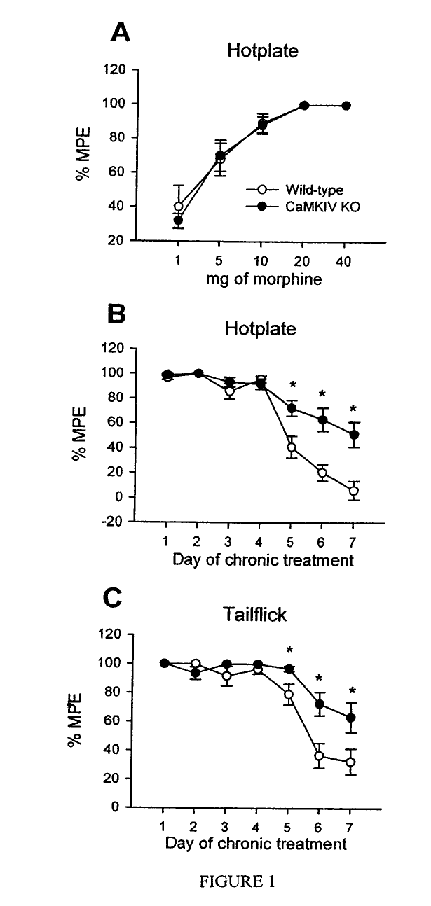

To determine if CaMKIV plays a role in the behavioral nociceptive responses to morphine, hot plate responses (55° C.) were measured after escalating doses of morphine in both CaMKIV KO and wild-type mice. Response latencies were similar between CaMKIV KO (n=8) and wild-type (n=8) mice at each morphine dose tested (FIG. 1A), suggesting that the deletion of CaMKIV did not affect morphine-induced analgesia. Hot plate response latencies were also recorded from CaMKIV KO and wild-type mice after repeated saline injections and were not altered with repeated hot plate exposure (data not shown).

CaMKIV KO Mice Develop Less Analgesic Tolerance after Chronic Morphine

To determine if CaMKIV plays a role in the behavioral responses to chronic morphine treatment, CaMKIV KO and wild-type mice were given daily injections of morphine (10 mg/kg, s.c.) and hot plate and tail flick response latencies were recorded. There was a significant effect of genotype in response latencies after chronic morphine treatment (p<0.001, FIGS. 1B and 1C). By the fifth morphine injection, CaMKIV KO mice displayed enhanced response latencies as compared to wild-type mice in both the hot plate and tail flick tests (FIGS. 1B and C). These results indicate that CaMKIV plays a role in the acquisition of opioid analgesic tolerance.

### Acute Morphine Tolerance in CaMKIV KO Mice

Acute analgesic tolerance can develop 24 hrs after an animal is challenged with a high dose of morphine (Bohn et al., 2000). In order to determine if CaMKIV is needed for the development of acute opioid tolerance, CaMKIV KO and wild-type mice received either a high dose (challenge dose, 100 mg/kg, s.c.) of morphine or an equivalent dose of saline on the first day of testing, and a moderate dose (10 mg/kg, s.c.) on the second day. Nociceptive response latencies were measured using two models of acute nociception (hot plate (55° C.) and tail flick test). Hot plate and tail flick response latencies were lower in both CaMKIV KO and wild-type mice that received morphine, as compared to saline, the day before, indicating similar degrees of acute tolerance between the two groups.

Morphine Induced Hyperlocomotion after Acute and Chronic Treatment

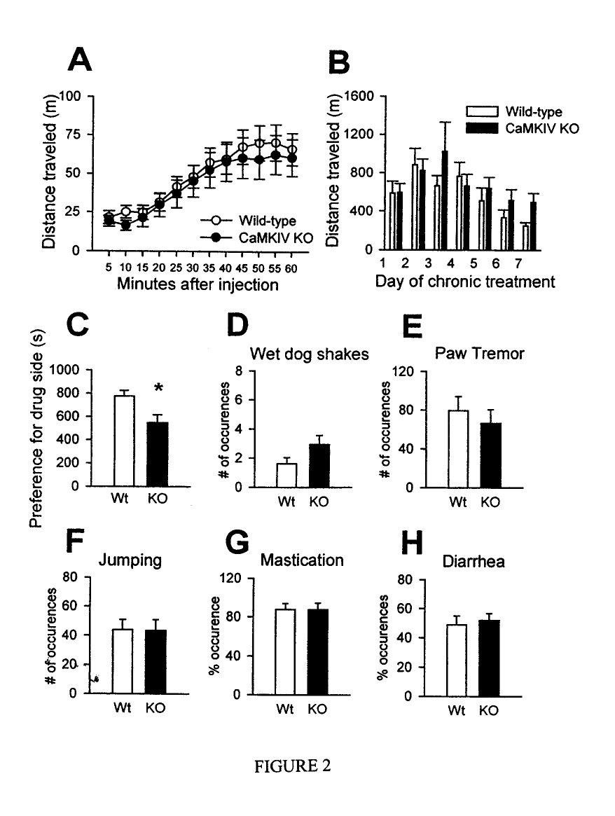

To determine if CaMKIV plays a role in morphine induced hyperlocomotor activity, locomotor activity was measured in an open field for 60 minutes following morphine administration. There was no difference in locomotion between CaMKIV KO (n=10) and wild-type (n=9) mice after a single injection of 10 mg/kg (s.c.) morphine (FIG. 2A). Additionally, locomotor activity was similar between CaMKIV KO and wild-type mice following chronic morphine administration (10 mg/kg, s.c. daily for seven days, FIG. 2B).

### Conditioned Place Preference to Morphine

The conditioned place preference paradigm was used to show that CREB mutant mice show a defect in the rewarding properties of morphine (Walters and Blendy, 2001). To determine if responses to the rewarding properties of morphine requires CaMKIV, CaMKIV KO (n=6) and wild-type (n=6) mice were tested in the conditioned place preference paradigm. CaMKIV KO mice spent significantly less time exploring the morphine-paired side of the chamber when compared to wild-type mice (p<0.02, FIG. 2C). CaMKIV KO and wild-type mice did not differ in their initial preference for either side of the chamber.

### Withdrawal Behaviors in CaMKIV KO Mice

Prolonged morphine use produces a state of physical dependence in addition to analgesic tolerance. To determine if CaMKIV plays a role in the development of opioid dependence, withdrawal behaviors were scored following chronic administration of morphine. Both wild-type and KO animals were injected with escalating doses of morphine for seven days after which physical withdrawal was precipitated with naloxone (1 mg/kg, s.c.). Stereotypic withdrawal behaviors (e.g., wet-dog shakes, paw tremors, and jumping) were scored for 30 minutes in both groups. There were no differences between CaMKIV KO (n=8) and wild-type (n=8) mice in any of the behaviors induced by the opioid receptor antagonist (see FIG. 2D-H). Although CaMKIV KO mice developed less analgesic tolerance to chronic administration of morphine, it appears that CaMKIV is not involved in morphine-induced physical dependence.

pCREB and MOR Expression in CaMKIV KO and WT Mice

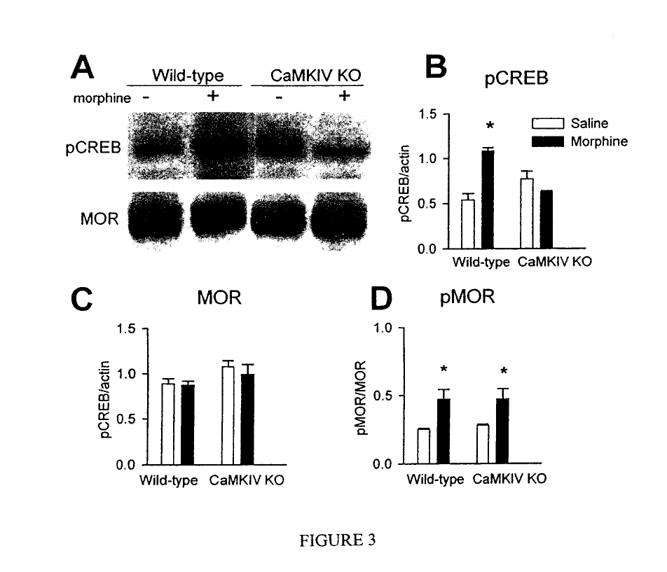

Studies have shown that chronic morphine treatment increases levels of pCREB expression in the spinal cord (Li and Clark, 1999). Here, pCREB expression was measured in the spinal cord of CaMKIV KO and WT mice before and after chronic morphine treatment. There was no difference in pCREB expression between CaMKIV KO and WT mice treated with saline. On the other hand, chronic morphine significantly increased pCREB expression in WT mice (p<0.001), while such an increase was absent in CaMKIV KO mice (FIG. 3A illustrates Western Blot results, graphically summarized in FIG. 3B). Since the MOR was shown to be activated through CREB-mediated pathways (Lee and Lee, 2003), MOR in CaMKIV KO mice was measured before and after chronic morphine. There was no difference in MOR expression between CaMKIV KO and WT mice either before or after morphine treatment (FIGS. 3A and C). Taken together, these results suggest that CaMKIV is involved in the morphine-induced upregulation of pCREB in the spinal cord and does not regulate MOR expression.

### Phosphorylation of the Mu Opioid Receptor

Since CaMKIV is detected in both the nuclei and cytoplasm of neurons (Jensen et al., 1991; Nakamura et al., 1995), its role in the development of opioid tolerance may extend beyond interactions with CREB. For example, phosphorylation of the MOR by several protein kinases (PKA, PKC, and CaMKII) can lead to desensitization, which is one of the proposed mechanisms for the development of opioid tolerance and dependence (Liu and Anand, 2001; Nestler, 1997). To determine if CaMKIV plays a role in this process, the serine phosphorylation state of the MOR was evaluated. The MOR was immunoprecipitated from spinal cord samples of CaMKIV KO and WT mice treated with either saline or chronic morphine to show that the deletion of CaMKIV did not affect serine phosphorylation of the MOR either before or after morphine treatment (FIG. 3D). These results suggest that the decrease in analgesic tolerance is not due to an alteration in the phosphorylation state of the MOR.

Expression of pCREB and MOR in the Spinal Cord of CaMKIV KO Mice

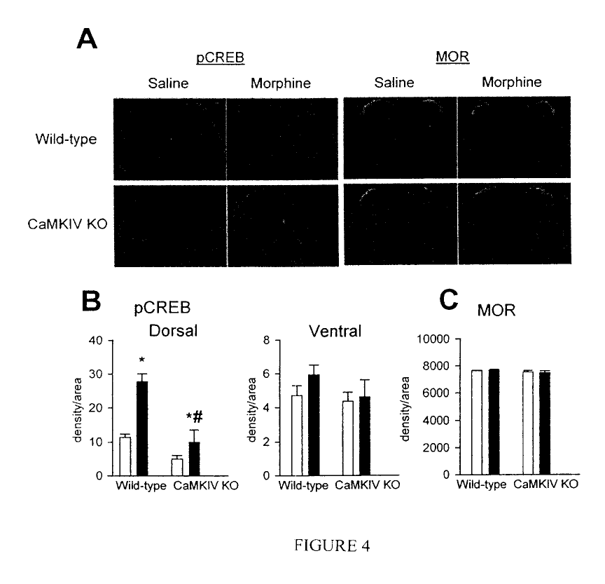

A previous study reported that the morphine-induced upregulation of pCREB was predominantly in lamina I-II of the dorsal horn (Li and Clark, 1999). Consistently, immunohistochemical staining for pCREB expression in the spinal cords of CaMKIV KO and WT mice chronically treated with saline or morphine revealed a significant role for CaMKIV in the morphine induced upregulation of pCREB in lamina I-II of the dorsal horn (FIGS. 4A and B). Morphine significantly increased pCREB in layer I and II of the dorsal horn in both CaMKIV KO and wild-type mice (p<0.001), but pCREB was significantly lower in knockout mice compared to wild-type treated with morphine (p<0.001) (“*” indicates significantly different from saline; “#” indicates significantly different from wild-type). As before, there was no significant difference in pCREB expression between knockout and WT mice treated with saline. Notably, there was no difference in the expression of pCREB in the ventral horn between saline and morphine treated CaMKIV KO and WT mice (FIG. 4B). Taken together, these results suggest that CaMKIV plays a role in the selective upregulation of pCREB in lamina I-II of the dorsal horn after chronic morphine administration. There was no difference in MOR expression between CaMKIV KO and WT mice treated with saline or morphine in lamina I and II of the dorsal horn (FIG. 4C).

### Mu Opioid Receptor Coupling in CaMKIV KO Mice

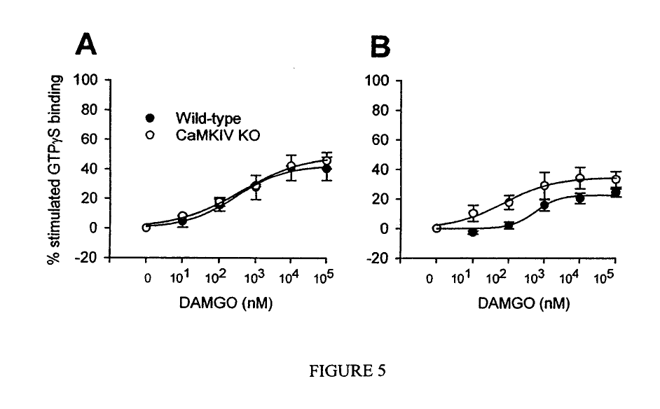

Uncoupling of G-proteins from the MOR is a hallmark of the cellular adaptations of opioid tolerance (Hyman, 1996; Nestler, 1997). G-protein binding was measured following activation by the highly selective MOR ligand DAMGO in the spinal cords of CaMKIV KO and WT mice after chronic morphine (10 mg/kg, s.c. daily for seven days). There was no difference in DAMGO-induced GTPγS binding between samples taken from CaMKIV KO and WT mice without morphine (FIG. 5A). However, GTPγS binding was significantly reduced in WT mice compared to CaMKIV KO mice chronically treated with morphine (FIG. 5B). This cellular adaptation in response to chronic morphine treatment appears to be decreased in the absence of CaMKIV.

CaMKIV Plays a Role in Morphine-Induced Reduction of Inhibitory Transmission after Prolonged Morphine Exposure

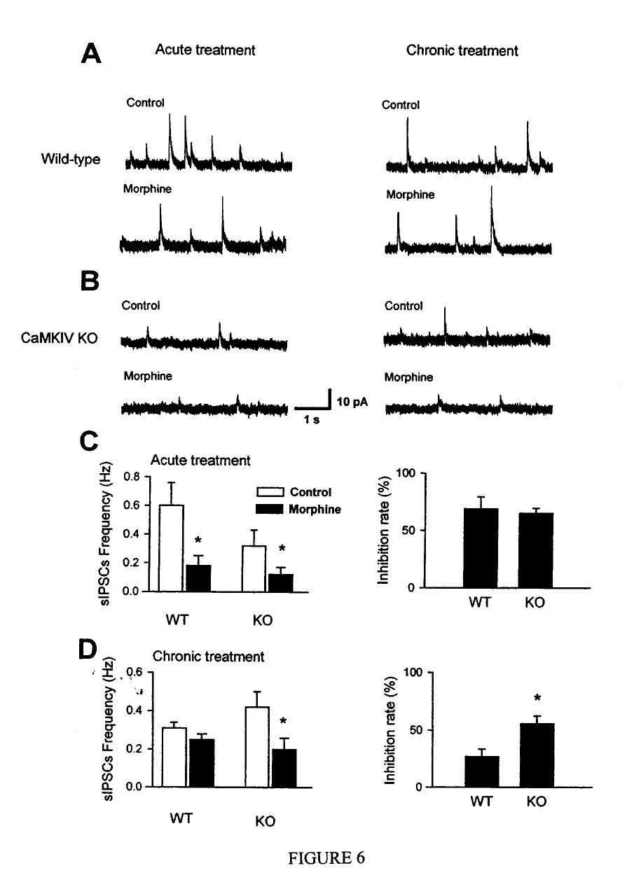

The analgesic effects of opioids are thought to be mediated by inhibiting GABAergic synaptic transmission in the periaqueductal grey and Locus Coeruleus (Vaughan, et al., 1997, Pan et al., 2002) and the activation of MORs was shown to suppress inhibitory transmission between spinal cord dorsal horn neurons (Kerchner and Zhuo, 2002). As shown herein, CaMKIV KO mice display less analgesic tolerance after chronic morphine; however, morphine-induced electrophysiological changes in inhibitory transmission are unknown. To determine if CaMKIV plays a role in morphine-induced changes in inhibitory synaptic transmission, sIPSCs were recorded from substantia gelatiosa (SG) neurons in lamina II of the dorsal horn. Acutely applied morphine dramatically decreased the frequency of sIPSCs in all SG neurons tested (WT, 68.9±10.4% n=7, p<0.05; CaMKIV KO, 66.2±4.0%, n=6, p<0.05, FIG. 6A-C). To determine if a prolonged pre-exposure to morphine (mimicking chronic morphine administration) would alter sIPSCs upon morphine application, slices were incubated in μM morphine for at least 1 hour before testing. As shown in FIG. 6D, morphine dramatically decreased the frequency of sIPSCs in CaMKIV KO mice by 55.7±6.9% (n=9, p<0.05), but did not decrease sIPSCs in WT mice (n=8, p>0.05). Taken together, these results suggest that pre-exposure to morphine is able to abolish the morphine-induced decreased in sIPSCs in WT mice but this effect is blocked in CaMKIV KO mice.

### Sub-Cellular Distribution of DORs in WT and CaMKIV KO Mouse Spinal Cord

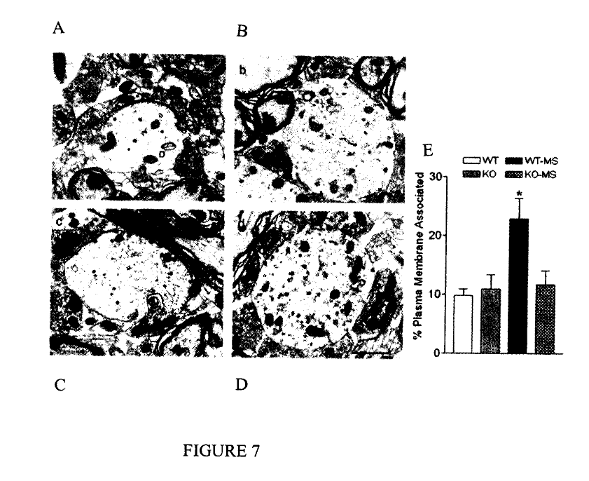

In the dorsal horn (lamina II-V) of untreated WT mice, silver-intensified immunogold particles, corresponding to immunoreactive DOR, were detected in association with axons, axon terminals, perikarya, and dendrites (FIGS. 7A and B). As previously demonstrated (Morinville et al., 2003), most immunoreactive DORs were intracellular rather than on the plasma membrane (FIGS. 7A and B). In the dorsal horn of the spinal cord of untreated CaMKIV KO mice, the subcellular distribution of immunoreactive DORs was similar to that observed in untreated WT mice (FIGS. 7 A and B). No significant difference was observed in the overall density of immunogold particles (per dendritic unit area) in WT as compared to knockout mice (data not shown). In addition, the proportion of immunogold particles associated with the plasma membrane was identical to that observed in WT mice (FIG. 7E). The lack of a difference in subcellular distribution between WT and CaMKIV KO mice suggests that under homeostatic conditions, the presence of CaMKIV is not required for the trafficking of DOR to the plasma membrane.

### Effect of CaMKIV Gene Knock-Out on Morphine-Induced Trafficking of DOR

Previous studies have established that prolonged morphine treatment induces trafficking of intracellular DOR to neuronal plasma membranes (Cahill et al., 2001; Morinville et al., 2003). To determine whether CaMKIV is involved in this targeting, the subcellular distribution of immunoreactive DOR was examined by electron microscopy. Pretreatment of WT mice with morphine (5, 8, 10, 15 mg/kg, s.c., every 12 h) did not produce any significant change in the number of immunogold particles detected per unit area of dorsal horn dendrites when compared to untreated WT mice (data not shown). However, the percentage of immunogold particles associated with neuronal plasma membranes was significantly increased (p<0.05, FIGS. 7A and C).

Morphine pretreatment of CaMKIV KO mice did not change the subcellular distribution of DORs within dendrites in the dorsal horn of the spinal cord (FIGS. 7B and D). Indeed, the percentage of immunogold particles associated with the plasma membrane was not significantly changed in morphine pretreated as compared to untreated KO mice (FIG. 7E, p>0.05). Chronic pretreatment of CaMKIV KO mice with morphine also failed to produce any change in the density of immunogold particles per unit area of dendrites when compared to either untreated KO mice or to morphine-pretreated WT mice (data not shown). These results conclusively demonstrate that morphine targets DORs to the plasma membrane through a mechanism involving CaMKIV.

### Discussion

Evidence for the role of a nuclear protein kinase, CamKIV, (which can phosphorylate CREB) in opioid tolerance, but not in physical dependence, is herein provided. While CaMKIV KO mice developed less analgesic tolerance after chronic morphine treatment, there was no difference in morphine-induced analgesia, acute opioid tolerance, physical dependence, or morphine-induced locomotor activity, as compared to wild-type mice. Additionally, G-protein uncoupling to the MOR, a hallmark of cellular opioid tolerance (Nestler, 2001a), was significantly less in CaMKIV KO mice compared to wild-type after chronic morphine treatment, while uncoupling was similar in saline treated controls (FIG. 6). Lastly, the increase in CREB phosphorylation seen in wild-type mice after chronic morphine administration was absent in CaMKIV KO mice. These results demonstrate for the first time the importance of the nuclear protein kinase, CamKIV, in both the behavioral and cellular adaptations following chronic morphine treatment.

### Experiment #2

### Transient Expression of CaMKIV and SiRNA Inhibition of CaMKIV

HEK293 cells were seeded into 96-well plates. The next day, cells were cotransfected with the CaMKIV construct, pGL3-CRE-firefly luciferase and pGL3-CMV-Renilla luciferase constructs using Lipofectamine 2000 reagent (Invitrogen, CA) according to the manufacturer's recommendation. The following day, cells were treated with fresh medium containing DMSO as vehicle control or 5 μM ionomycin (Sigma, Mo.) in the presence or absence of KN62 or KN93 (Sigma, MO) at serial concentrations. Cells were harvested 16 h later. Luciferase activity was assayed with cell lysis by using the dual-luciferase reporter assay system (Promega, WI) according to the manufacturer's protocol.

### Primary Culture of Cortical Neurons

Cortical neurons were prepared from postnatal day 0 (P0) mice. All procedures were reviewed and approved by the Institutional Animal Care and Use Committee at University of Toronto, in accordance with the guidelines of the Canadian Council on Animal Care. The Cerebral cortexes were dissected, minced, and trypsinized for 15 min using 0.125% trypsin (Invitrogen, CA). Cells were seeded at the density of 4˜5×10⁵ cells/cm² onto 60 mm dishes or 96-well plates precoated with 50 μg/ml poly-D-lysine (Sigma, Mo.) in water, and grown in Neurobasal-A medium (Invitrogen, CA) supplemented with B27 and GlutaMax (Invitrogen, CA). The cultures were incubated at 37° C. in 95% air, 5% carbon dioxide with 95% humidity.

siRNA Preparation and Transfection

The two selected siRNA sequences for mouse CaMKIV were as follows:

sequence 1: sense 5′-GCAUGAUAUGCACUAAUAGtt-3′, antisense 5′-CUAUUAGUGCAUAUCAUGCtt-3′ and
sequence 2: sense 5′-CGGCUGACUACAUUUCAAGtt-3′, antisense 5′-CUUGAAAUGUAGUCAGCCGtt-3′, respectively (Ambion, Tex.). The annealed siRNA was reconstituted at the concentration of 100 μM for transfection. Lipofectamine 2000 (Invitrogen) was mixed with the siRNA according to the manufacturer's instructions and added to cortical neurons that had been cultured for 5 d. The siRNA concentration for transfection is 30 nM. After 6-h incubation, the transfection complex was replaced with Neurobasal-A medium. Cells were harvested and analyzed 48 h after transfection.

### Western Blot Analysis.

The cultured cortical neurons were harvested and homogenized in lysis buffer containing proteinase inhibitor cocktail (Sigma, Mo.). Protein was quantified by Bradford assay, and electrophoresis of equal amounts of total protein was performed on SDS-polyacrylamide gels. Separated proteins were transferred to polyvinylidene fluoride membranes at 4° C. overnight. Membranes were probed with 1:2000 dilution of anti-CaMKIV (Santa Cruz, Calif.). The membranes were then incubated in the horseradish peroxidase-coupled secondary antibody diluted 1:3000 for 1 h followed by enhanced chemiluminescence detection of the proteins with Western lightning chemiluminescence reagent plus (Perkin Elmer Life sciences, MA) according to the manufacturer's instructions. To verify equal loading, membranes were also probed with anti-actin antibody (Sigma, Mo.). The density of immunoblots was measured using NIH ImageJ software.

### Results

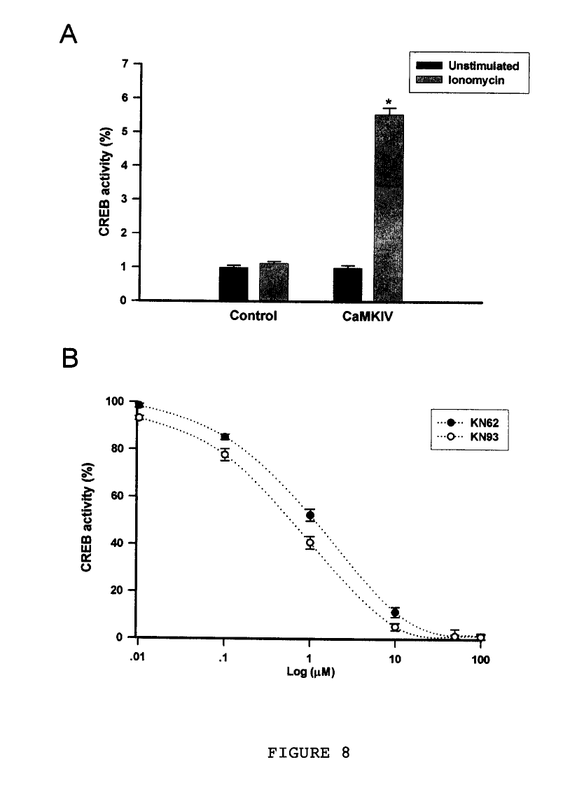

The dose-dependent inhibitory effect of KN62 and KN93 on CaMKIV transiently expressed in HEK293 cells was determined. CaMKIV can activate CREB and stimulate CREB-mediated transcription through the direct phosphorylation of CREB on Ser-133. To measure the CaMKIV activity by CREB activity assay, the CaMKIV expression plasmids and a CREB luciferase reporter system were cotransfected into HEK293 cells in this study. The calcium ionophore ionomycin treatment can lead to an increase in intracellular Ca²⁺ and Ca²⁺/CaM levels, which influence CaMKIV activity in cells. Ionomycin (5 μM) can dramatically increase CREB activity in HEK293 cells expressing CaMKIV. The CREB activity is 5.5±0.2 times that of unstimulated cells (p<0.0001, compared to the unstimulated cells, n=4 wells, FIG. 8A).

The effect of KN62 (4-[(25)-2-[(5-isoquinolinylsulfonyl)methylamino]-3-oxo-3-(4-phrenyl-1-piperazinyl)propyl]phenyl isoquinolinesulfonic acid ester) and KN-93 (N-[2-[[[3-(4-Chlorophenyl)-2-propenyl]methylamino]methyl]phenyl]-N-(2-hydroxyethyl)-4-methoxybenzenesulphonamide) on CaMKIV activity was also tested using this system. Both of these chemicals exhibited a dose-dependent inhibitory effect on CaMKIV transiently expressed in HEK293 cells with CREB activity of 98.4±0.8%, 85.3±1.2%, 55.5±2.5%, 11.5±2.6%, 1.3±2.9% and 1.2±1.4% of the control level for KN62, and 93.1±0.9%, 77.7±2.5% 40.8±2.7%, 5.3±1.5% 1.3±0.7% and 1.1±0.9% of the control level for KN93 at concentrations of 0.01 μM, 0.1 μM, 1.0 μM, 10 μM, 50 μM and 100 μM, respectively. The IC50 for KN62 and KN93 is 1.06 μM and 0.58 μM, respectively (n=4 wells, FIG. 8B).

The Effect of siRNA on CaMKIV Expression in Cultured Cortical Neurons

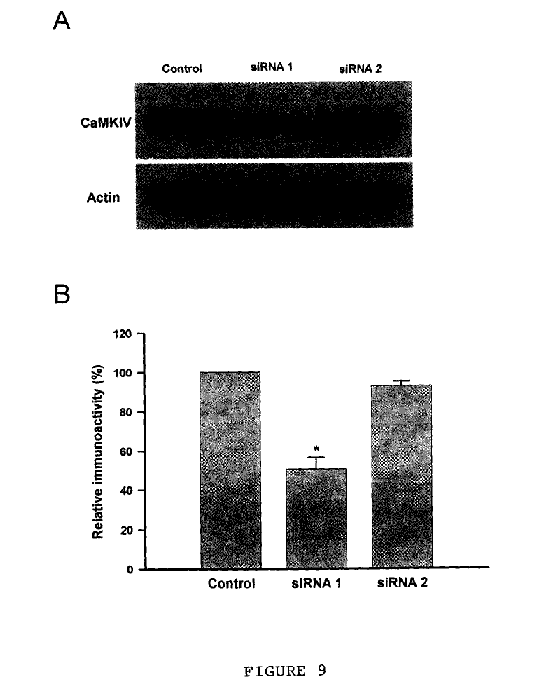

To knock down the expression of CaMKIV, two siRNA sequences specific for mouse CaMKIV were used to determine the effect on the expression of CaMKIV in cultured cortical neurons. 48 hours after transfection of siRNA, it was found that sequence 1 could efficiently knock down the expression of CaMKIV as shown by western blot, the expression level of CaMKIV is 50.6±5.9% of the control (p<0.01 compared to the control, n=3 experiments), whereas sequence 2 could not knock down the expression of CaMKIV ((n=3 experiments, FIG. 9)

### REFERENCES

Barrot, M., Olivier, J. D., Perrotti, L. I., DiLeone, R. J., Berton, O., Eisch, A. J., Impey, S., Storm, D. R., Neve, R. L., Yin, J. C., et al. (2002). CREB activity in the nucleus accumbens shell controls gating of behavioral responses to emotional stimuli. Proc Natl Acad Sci USA 99, 11435-11440.
Blendy, J. A., and Maldonado, R. (1998). Genetic analysis of drug addiction: the role of cAMP response element binding protein. J Mol Med 76, 104-110.
Bohn, L. M., Gainetdinov, R. R., Lin, F. T., Lefkowitz, R. J., and Caron, M. G. (2000). Mu-opioid receptor desensitization by beta-arrestin-2 determines morphine tolerance but not dependence. Nature 408, 720-723.
Bohn, L. M., Lefkowitz, R. J., Gainetdinov, R. R., Peppel, K., Caron, M. G., and Lin, F. T. (1999). Enhanced morphine analgesia in mice lacking beta-arrestin 2. Science 286, 2495-2498.
Carlezon, W. A., Jr., Thome, J., Olson, V. G., Lane-Ladd, S. B., Brodkin, E. S., Hiroi, N., Duman, R. S., Neve, R. L., and Nestler, E. J. (1998). Regulation of cocaine reward by CREB. Science 282, 2272-2275.
Chen, S. R., and Pan, H. L. (2003). Up-regulation of spinal muscarinic receptors and increased antinociceptive effect of intrathecal muscarine in diabetic rats. J Pharmacol Exp Ther 307, 676-681.
Christie, M. J., Williams, J. T., and North, R. A. (1987). Cellular mechanisms of opioid tolerance: studies in single brain neurons. Mol Pharmacol 32, 633-638.
Deisseroth, K., Bito, H., and Tsien, R. W. (1996). Signaling from synapse to nucleus: postsynaptic CREB phosphorylation during multiple forms of hippocampal synaptic plasticity. Neuron 16, 89-101.
Deisseroth, K., Heist, E. K., and Tsien, R. W. (1998). Translocation of calmodulin to the nucleus supports CREB phosphorylation in hippocampal neurons. Nature 392, 198-202.
Eitan, S., Bryant, C. D., Saliminejad, N., Yang, Y. C., Vojdani, E., Keith, D., Jr., Polakiewicz, R., and Evans, C. J. (2003). Brain region-specific mechanisms for acute morphine-induced mitogen-activated protein kinase modulation and distinct patterns of activation during analgesic tolerance and locomotor sensitization. J Neurosci 23, 8360-8369.
Gao, C., Chen, L., Tao, Y., Chen, J., Xu, X., Zhang, G., and Chi, Z. (2004). Colocalization of phosphorylated CREB with calcium/calmodulin-dependent protein kinase IV in hippocampal neurons induced by ohmfentanyl stereoisomers. Brain Res 1024, 25-33.
Ginty, D. D. (1997). Calcium regulation of gene expression: isn't that spatial? Neuron 18, 183-186.
Guitart, X., Thompson, M. A., Mirante, C. K., Greenberg, M. E., and Nestler, E. J. (1992). Regulation of cyclic AMP response element-binding protein (CREB) phosphorylation by acute and chronic morphine in the rat locus coeruleus. J Neurochem 58, 1168-1171.
Hyman, S. E. (1996). Shaking out the cause of addiction. Science 273, 611-612.
Hyman, S. E., and Malenka, R. C. (2001). Addiction and the brain: the neurobiology of compulsion and its persistence. Nat Rev Neurosci 2, 695-703.
Jensen, K. F., Ohmstede, C. A., Fisher, R. S., and Sahyoun, N. (1991). Nuclear and axonal localization of Ca2+/calmodulin-dependent protein kinase type Gr in rat cerebellar cortex. Proc Natl Acad Sci USA 88, 2850-2853.
Johnson, E. C., Bohn, L. M., Barak, L. S., Birse, R. T., Nassel, D. R., Caron, M. G., and Taghert, P. H. (2003). Identification of Drosophila neuropeptide receptors by G protein-coupled receptors-beta-arrestin2 interactions. J Biol Chem 278, 52172-52178.
Kandel, E. R. (2001). The molecular biology of memory storage: a dialogue between genes and synapses. Science 294, 1030-1038.
Kang, H., Sun, L. D., Atkins, C. M., Soderling, T. R., Wilson, M. A., and Tonegawa, S. (2001). An important role of neural activity-dependent CaMKIV signaling in the consolidation of long-term memory. Cell 106, 771-783.
Lee, P. W., and Lee, Y. M. (2003). Transcriptional regulation of mu opioid receptor gene by cAMP pathway. Mol Pharmacol 64, 1410-1418.
Li, X., and Clark, J. D. (1999). Morphine tolerance and transcription factor expression in mouse spinal cord tissue. Neurosci Lett 272, 79-82.
Liu, J. G., and Anand, K. J. (2001). Protein kinases modulate the cellular adaptations associated with opioid tolerance and dependence. Brain Res Brain Res Rev 38, 1-19.
Maldonado, R., Blendy, J. A., Tzavara, E., Gass, P., Rogues, B. P., Hanoune, J., and Schutz, G. (1996). Reduction of morphine abstinence in mice with a mutation in the gene encoding CREB. Science 273, 657-659.
Matthews, R. P., Guthrie, C. R., Wailes, L. M., Zhao, X., Means, A. R., and McKnight, G. S. (1994). Calcium/calmodulin-dependent protein kinase types II and IV differentially regulate CREB-dependent gene expression. Mol Cell Biol 14, 6107-6116.
Mestek, A., Hurley, J. H., Bye, L. S., Campbell, A. D., Chen, Y., Tian, M., Liu, J., Schulman, H., and Yu, L. (1995). The human mu opioid receptor: modulation of functional desensitization by calcium/calmodulin-dependent protein kinase and protein kinase C. J Neurosci 15, 2396-2406.
Nakamura, Y., Okuno, S., Sato, F., and Fujisawa, H. (1995). An immunohistochemical study of Ca2+/calmodulin-dependent protein kinase IV in the rat central nervous system: light and electron microscopic observations. Neuroscience 68, 181-194.
Narita, M., Mizoguchi, H., and Tseng, L. F. (1995). Inhibition of protein kinase C, but not of protein kinase A, blocks the development of acute antinociceptive tolerance to an intrathecally administered mu-opioid receptor agonist in the mouse. Eur J Pharmacol 280, R1-3.
Nestler, E. J. (1997). Molecular mechanisms of opiate and cocaine addiction. Curr Opin Neurobiol 7, 713-719.
Nestler, E. J. (2001a). Molecular neurobiology of addiction. Am J Addict 10, 201-217.
Nestler, E. J. (2001b). Neurobiology. Total recall-the memory of addiction. Science 292, 2266-2267.
Nestler, E. J. (2002). Common molecular and cellular substrates of addiction and memory. Neurobiol Learn Mem 78, 637-647.
Nitsche, J. F., Schuller, A. G., King, M. A., Zengh, M., Pasternak, G. W., and Pintar, J. E. (2002). Genetic dissociation of opiate tolerance and physical dependence in delta-opioid receptor-1 and preproenkephalin knock-out mice. J Neurosci 22, 10906-10913.
Sim, L. J., Selley, D. E., Dworkin, S. I., and Childers, S. R. (1996). Effects of chronic morphine administration on mu opioid receptor-stimulated [35S]GTPgammaS autoradiography in rat brain. J Neurosci 16, 2684-2692.
Soderling, T. R. (1999). The Ca-calmodulin-dependent protein kinase cascade. Trends Biochem Sci 24, 232-236.
Terman, G. W., Jin, W., Cheong, Y. P., Lowe, J., Caron, M. G., Lefkowitz, R. J., and Chavkin, C. (2004). G-protein receptor kinase 3 (GRK3) influences opioid analgesic tolerance but not opioid withdrawal. Br J Pharmacol 141, 55-64.
Ueda, H., Inoue, M., and Matsumoto, T. (2001). Protein kinase C-mediated inhibition of mu-opioid receptor internalization and its involvement in the development of acute tolerance to peripheral mu-agonist analgesia. J Neurosci 21, 2967-2973.
Walters, C. L., and Blendy, J. A. (2001). Different requirements for cAMP response element binding protein in positive and negative reinforcing properties of drugs of abuse. J Neurosci 21, 9438-9444.
Walters, C. L., Godfrey, M., Li, X., and Blendy, J. A. (2005). Alterations in morphine-induced reward, locomotor activity, and thermoregulation in CREB-deficient mice. Brain Res 1032, 193-199.
Wang, Z. J., Tang, L., and Xin, L. (2003). Reversal of morphine antinociceptive tolerance by acute spinal inhibition of Ca(2+)/calmodulin-dependent protein kinase II. Eur J Pharmacol 465, 199-200.
Wei, F., Qiu, C. S., Liauw, J., Robinson, D. A., Ho, N., Chatila, T., and Zhuo, M. (2002). Calcium calmodulin-dependent protein kinase IV is required for fear memory. Nat Neurosci 5, 573-579.
Zeitz, K. P., Malmberg, A. B., Gilbert, H., and Basbaum, A. I. (2001). Reduced development of tolerance to the analgesic effects of morphine and clonidine in PKC gamma mutant mice. Pain 94, 245-253.

We claim:

1. A method of treating chronic pain in a mammal comprising the steps of inhibiting CaMKIV in the mammal and administering to the mammal a therapeutically effective amount of an opioid.

2. A method as defined in claim 1, wherein CaMKIV is inhibited by administration of at least one compound selected from the group consisting of a chemical CaMKIV inhibitor, an antisense oligonucleotide and siRNA fragments.

3. A method as defined in claim 2, wherein CaMKIV is inhibited by siRNA fragments.

4. A method as defined in claim 3, wherein the siRNA fragments are: sense 5′-GCAUGAUAUGCACUAAUAGtt-3′ and antisense 5′-CUAUUAGUGCAUAUCAUGCtt-3′.

5. A method as defined in claim 1, wherein the opioid is an mu receptor agonist.

6. A method as defined in claim 1, wherein the opioid is selected from the group consisting of full agonist opioids, partial agonist opioids and mixed agonist-antagonist opioids.

7. A method as defined in claim 1, wherein the opioid is selected from the group consisting of morphine, hydromorphone, codeine, oxycodone, hydrocodone, methadone, levorphanol, fentanyl, buprenorphene, pentazocine, butorphanol tartrate, dezocine, and nalbuphine hydrochloride.

8. A method of treating chronic pain in a mammal comprising administration of an opioid in combination with an inhibitor of CaMKIV.

9. A pharmaceutical composition comprising a therapeutically effective amount of an opioid in combination with a CaMKIV inhibitor.

10. A composition as defined in claim 9, wherein the CaMKIV inhibitor is selected from the group consisting of a chemical CaMKIV inhibitor, an antisense oligonucleotide and siRNA fragments.

11. A composition as defined in claim 10, wherein the CaMKIV inhibitor is siRNA fragments.

12. A composition as defined in claim 10, wherein the siRNA fragments are: sense 5′-GCAUGAUAUGCACUAAUAGtt-3′ and antisense 5′-CUAUUAGUGCAUAUCAUGCtt-3′.

13. A composition as defined in claim 8, wherein the opioid is selected from the group consisting of full agonist opioids, partial agonist opioids and mixed agonist-antagonist opioids.

14. A composition as defined in claim 8, wherein the opioid is an mu receptor agonist.

15. A composition as defined in claim 13, wherein the opioid is selected from the group consisting of morphine, hydromorphone, codeine, oxycodone, hydrocodone, methadone, levorphanol, fentanyl, buprenorphene, pentazocine, butorphanol tartrate, dezocine, and nalbuphine hydrochloride.

16. An article of manufacture comprising packaging material and a pharmaceutical composition, wherein the composition comprises a therapeutically effective amount of an opioid in combination with a CaMKIV inhibitor, and wherein the packaging material is labeled to indicate that the composition is useful to treat chronic pain.

17. An article of manufacture as defined in claim 16, wherein the opioid is selected from the group consisting of full agonist opioids, partial agonist opioids and mixed agonist-antagonist opioids.

18. An article of manufacture as defined in claim 16, wherein the opioid is selected from the group consisting of morphine, hydromorphone, codeine, oxycodone, hydrocodone, methadone, levorphanol, fentanyl, buprenorphene, pentazocine, butorphanol tartrate, dezocine, and nalbuphine hydrochloride.

19. An article of manufacture as defined in claim 16, wherein the CaMKIV inhibitor is selected from the group consisting of a chemical CaMKIV inhibitor, an antisense oligonucleotide and siRNA fragments.

20. An article of manufacture as defined in claim 16, wherein the opioid is an mu receptor agonist.

21. The siRNA fragments, GCAUGAUAUGCACUAAUAGtt-3′ and 5′-CUAUUAGUGCAUAUCAUGCtt-3′.
